(19) United States
(12) Patent Application Publication
(10) Pub. No.: US 2016/0067237 A1
(43) Pub. Date: Mar. 10, 2016

(54) Sensory Stimulation for Cessation of Eating

(71) Applicant: WILLPOWER LABS, INC., San Francisco, CA (US)

(72) Inventor: Mark W. Bernstein, San Francisco, CA (US)

(73) Assignee: Willpower Labs, Inc., San Francisco, CA (US)

(21) Appl. No.: 14/890,128

(22) PCT Filed: May 7, 2014
(86) PCT No.: PCT/US14/37113

§ 371 Date: Nov. 9, 2015

Related U.S. Application Data
(60) Provisional application No. 61/821,788, filed on May 10, 2013.

(51) Int. Cl. A61K 31/4525; A61K 9/28; A61K 31/164; A61K 31/26; A61K 31/165; A61K 31/12; A61K 31/085; A23L 1/29; A61K 31/045

(52) U.S. Cl. CPC A61K 31/4525; A23L 1/293; A61K 9/2806; A61K 31/164; A61K 31/045; A61K 31/165; A61K 31/12; A61K 31/085; A61K 31/26; A23V 2002/00

(57) ABSTRACT

Methods and compositions are provided for modifying the eating behavior of an individual. The compositions provide an oral stimulatory effect such as tingling, cooling, and/or warming. Consumption of the compositions after eating a desired amount of food may habituate the individual to ending a meal and serve to cue cessation of eating, assisting the individual with ending a meal at a time point before a signal of satiety has reached the brain. Consumption of the composition in lieu of snacking between meals may assist the individual with avoiding consumption of snacks.

### CROSS-REFERENCE TO RELATED APPLICATIONS

This application claims the benefit of U.S. Provisional Application No. 61/821,788, filed on May 10, 2013, which is incorporated herein by reference in its entirety.

### FIELD OF THE INVENTION

The invention relates to methods and compositions for modifying the eating behavior of individuals and for cueing the cessation of eating after a desired amount of food has been consumed.

### BACKGROUND

Seventy percent of Americans are either overweight or obese. The World Health Organization calculates that globally, there are 1.4 billion overweight people. The greatest cause of this weight crisis is overeating. Individuals often have trouble regulating their eating because they can't end their meals after a healthy amount of food has been consumed. Individuals may not know in “real time” when they are full and have eaten enough food because it can take approximately 10-30 minutes for a signal of satiety to reach the brain from the stomach and the intestine. An individual may overindulge in the time frame between consumption of a healthy amount of food and the time at which the signal of satiety reaches the brain. It would be desirable to provide tools to assist individuals in modifying overeating behavior.

### BRIEF SUMMARY OF THE INVENTION

Methods and compositions are provided for modifying the behavior of an individual by cueing cessation of eating before an individual has consumed more than a desired amount of food, e.g., an amount of food, such as the minimum amount of food, that would cause satiety in the individual with the passage of time.

In one aspect, a method is provided for modifying eating behavior in an individual. After consuming an amount of food, the individual orally consumes a composition that causes oral sensation(s) that (i) make consuming a further amount of food undesirable and/or (ii) cue cessation of eating when consumed after a number of meals sufficient to provide a habitual sense of meal completion.

In some embodiments of the methods disclosed herein, the amount of food consumed before consumption of the composition includes food in portion sizes that would create satiation in the individual with the passage of time, for example, the minimum amount of food that would cause satiation in the individual with the passage of time. In some embodiments, the individual acquires a habitual sense of meal completion after consuming the composition after each meal for a period of time, e.g., for a period of time sufficient to acquire the habit. In some embodiments, the individual acquires a habitual sense of meal completion after consuming the composition after certain meals (e.g., intermittently), either after the same meal(s) each day or on some days, or after different meals on the same or different day(s). In some embodiments, the sense of meal completion (e.g., lack of desire to consume more food) lasts for at least about 10 minutes, at least about 15 minutes, at least about 20 minutes, at least about 25 minutes, or at least about 30 minutes, and/or for a length of time that is sufficient for the individual to acquire a feeling of satiety without further consumption of food.

In some embodiments of the methods disclosed herein, the composition that causes oral sensation(s) stimulates the trigeminal nerve, e.g., the third division of the trigeminal nerve. In some embodiments, stimulation of the trigeminal nerve cues the cessation of eating in an individual in whom a habitual sense of meal completion and/or a lack of desire to consume more food has been acquired by repeated consumption of the composition (e.g., trigeminal stimulation) after meals.

In some embodiments, the composition that causes oral sensation(s) is consumed by an individual to avoid consuming a snack and/or to avoid eating between meals. The composition creates sensation(s) in the mouth of the individual that make eating (e.g., consuming a snack, for example, a high calorie snack) undesirable. For example, repeated consumption of the composition at the end of meals may create a cue that eating is unnecessary or undesirable when the individual encounters the taste and experience of the oral sensation(s) in the composition. When the individual then consumes the composition when they have an urge to snack, encountering the oral sensation(s) to which they have become habituated as signaling the end of eating may provide the cue to avoid eating and may create a distraction or transference from the desire to snack. This may help the individual avoid unhealthy and/or high calorie foods between meals. In some embodiments, the oral composition is consumed in place of a snack, providing a deterrent to snacking.

In some embodiments of the methods disclosed herein, the oral sensation(s) caused by the composition includes at least two sensations selected from tingling, cooling, and warming. In one embodiment, the oral sensation includes tingling plus at least one sensation selected from cooling and warming. In some embodiments, the oral sensation comprises or consists of tingling and cooling. In some embodiments, the oral sensation includes a tingling sensation that is provided by at least one compound selected from spilanthol, isobutylalkylamide, and hydroxyl-alpha sanshool. In some embodiments, the oral sensation includes a cooling sensation that is provided by menthol or a derivative thereof. In some embodiments, the oral sensation includes a warming sensation that is provided by at least one compound selected from capsaicin, a capsaicinoid, shogaol, a zingerone, a gingergol, eugenol, piperine, and allyl isothiocyanate.

In some embodiments of the methods disclosed herein, the composition is formulated as a candy (e.g., a hard candy), a lozenge, a toothpaste, a chewing gum, a spray, a dissolving strip, a toothpick, a dental floss, a quick shot, a tea, a dissolving drop, a beverage, or a dessert. In one embodiment, the composition is formulated as a candy or lozenge with an outer layer or compound coating that includes at least one sweetener compound that provides a sweet (e.g., dessert-like) flavor.

In some embodiments of the methods disclosed herein, the composition further includes at least one flavoring that does not provide a tingling, cooling, or warming sensation. Examples of such flavorings include, but are not limited to, chocolate, cocoa, coffee, cinnamon, and lime and/or other fruit flavors.

In some embodiments of the methods disclosed herein, the composition includes an outer layer or coating that contains at least one sweetener compound and at least one flavoring. In one embodiment, the outer layer or coating contains sweetened chocolate.

In some embodiments of the methods disclosed herein, the composition further includes at least one additive (e.g., at least one non-flavoring additive) that does not provide a tingling, cooling, or warming sensation. Examples of such additives include, but are not limited to, caffeine, vitamins, calcium, and probiotics.

In one embodiment, a method is provided for promoting weight loss in an individual in need thereof and/or for treating obesity. The method includes orally consuming a composition as disclosed herein after the individual has consumed a desired amount of food, wherein the individual ceases eating earlier than they would have in the absence of consumption of the composition, and wherein consuming the composition after eating causes the individual to consume less food and lose weight. In some embodiments, the desired amount of food consumed before consuming the composition includes a meal with food in portion sizes that would create satiation in the individual with the passage of time, for example, the minimum amount of food that would cause satiation in the individual with the passage of time. In some embodiments, the individual acquires a habitual sense of meal completion after consumption of the composition after each meal for a period of time e.g., for a period of time sufficient to acquire the habit. In some embodiments, the individual acquires a habitual sense of meal completion after consuming the composition after certain meals (e.g., intermittently), either after the same meal(s) each day or on some days, or after different meals on the same or different day(s). In some embodiments, the sense of meal completion (e.g., lack of desire to consume more food) lasts for at least about 10 minutes, at least about 15 minutes, at least about 20 minutes, at least about 25 minutes, or at least about 30 minutes, and/or for a length of time that is sufficient for the individual to acquire a feeling of satiety without further consumption of food.

In one embodiment, a method is provided for maintaining an individual's weight within a desired weight range. The method includes orally consuming a composition as disclosed herein after the individual has consumed a desired amount of food, for example, the minimum amount of food that would cause satiation in the individual with the passage of time, wherein the oral administration is continued after meals for a period of time until a habit is established in the individual that a meal is completed after the composition has been consumed, and wherein establishment of the habit causes the individual to maintain their weight in a desired weight range. In some embodiments, the individual acquires a habitual sense of meal completion after consumption of the composition after each meal for a period of time e.g., for a period of time sufficient to acquire the habit. In some embodiments, the individual acquires a habitual sense of meal completion after consuming the composition after certain meals (e.g., intermittently), either after the same meal(s) each day or on some days, or after different meals on the same or different day(s). In some embodiments, the sense of meal completion (e.g., lack of desire to consume more food) lasts for at least about 10 minutes, at least about 15 minutes, at least about 20 minutes, at least about 25 minutes, or at least about 30 minutes, and/or for a length of time that is sufficient for the individual to acquire a feeling of satiety without further consumption of food.

In another aspect, a composition is provided that may be used in a method as disclosed herein for signaling completion of a meal. The composition includes compounds that provide at least two oral sensations selected from tingling, cooling, and warming when consumed orally by an individual. In some embodiments, the oral sensation comprises tingling plus at least one sensation selected from cooling and warming. In one embodiment, the oral sensation includes tingling and cooling. In another embodiment, the oral sensation includes tingling and warming. In another embodiment, the oral sensation includes tingling, cooling, and warming.

In some embodiments, the composition is formulated for stimulation of the trigeminal nerve, e.g., the third division of the trigeminal nerve, when consumed orally by an individual.

In some embodiments, the composition includes at least one compound that provides a tingling sensation when consumed orally by an individual, including but not limited to, spilanthol, isobutylalkylamide, or hydroxyl-alpha sanshool. In some embodiments, the composition includes at least one compound that provides a cooling sensation when consumed orally by an individual, including but not limited to, menthol or a derivative thereof. In some embodiments, the composition includes at least one compound that provides a warming sensation when consumed orally by an individual, including but not limited to, capsaicin, a capsaicinoid, shogaol, a zingerone, a gingergol, eugenol, piperine, or allyl isothiocyanate.

A composition as disclosed herein may be formulated in any form that is suitable for oral consumption by an individual, including but not limited to, a candy (e.g., a hard candy), a lozenge, a toothpaste, a chewing gum, a spray, a dissolving strip, a toothpick, a dental floss, a quick shot, a tea, a dissolving drop, a beverage, or a dessert. In one embodiment, the composition is formulated as a candy or lozenge with layers. In one embodiment, the composition is formulated as a candy or lozenge with an outer layer that includes at least one sweetener compound that provides a sweet (e.g., dessert-like) flavor when orally consumed.

In some embodiments, a composition as disclosed herein includes at least one flavoring that does not provide a tingling, cooling, or warming sensation. Examples of such flavorings include, but are not limited to, chocolate, cocoa, coffee, cinnamon, and lime and/or other fruit flavors.

In some embodiments, an outer layer or coating contains at least one sweetener compound and at least one flavoring. In one embodiment, the outer layer or coating contains sweetened chocolate.

In some embodiments, a composition as disclosed herein includes at least one additive (e.g., at least one non-flavoring additive) that does not provide a tingling, cooling, or warming sensation. Examples of such additives include, but are not limited to, caffeine, vitamins, calcium, and probiotics.

In another aspect, a system for signaling meal completion and/or for avoiding snacking between meals is provided. The system includes a composition as described herein that may be consumed after a meal to signal meal completion. The system may optionally further include instructions for use of the composition in a method for modifying eating behavior, a method for weight loss, a method for treating obesity, a method for maintaining a desired body weight and/or a method for avoiding undesired snacking, such as snacking on unhealthy or high calorie foods between meals. The system may be in the form of a kit that contains one or multiple portions of the composition in packaging, optionally with instructions for use in a method as described herein.

### DETAILED DESCRIPTION

Methods and compositions are disclosed herein for interrupting an individual's eating behavior, making continued eating less appealing or undesirable. In some embodiments, the methods and systems disclosed herein help an individual stop food consumption before a natural biological signal of satiety reaches the individual's brain.

A full stomach is only partially responsible for causing an individual to feel satisfied after a meal. Satiety, the sensation that an individual has had enough to eat, results from a combination of hormonal and neurological signals reaching the brain from both the stomach and the gastrointestinal tract. After food reaches the stomach, it must traverse the intestine via peristalsis to arrive at cells in the ileum and colon that release Peptide YY, which circulates in the blood stream, travels to the brain, and signals the hypothalamus that enough food has been consumed. It takes approximately 10 minutes to 30 minutes for food to reach the intestinal cells that release Peptide YY and for the Peptide YY signal to be received by the hypothalamus, the timing of which may vary depending on the individual and the type of food consumed.

The terms “hunger” and “satiety” are conventional terms in the art used to describe an individual's drive to obtain and ingest food. These neurophysiological responses are controlled in part by nerve connections between the stomach and duodenum and brain, as well as by circulating hormones that affect an individual's perceptions of hunger or satiety. Other factors that affect appetite include psychological factors, such as eating for pleasure, eating in a social context, and physical factors, such as blood sugar levels, dehydration, and physical activity.

In the methods disclosed herein, a “meal completion system” is provided that assists an individual in ending a meal after a desired amount of food has been consumed. The system may be used to modify an individual's eating behavior to discourage overeating. By using the system over a period of time, a habit may be developed such that when the individual tastes the composition and encounters the oral sensation(s) provided by the composition, this cues the individual that the meal is complete and eating is finished, thus reducing or eliminating overeating by the individual.

The compositions disclosed herein effect the tactile nervous system in the mouth of an individual, e.g., via the trigeminal nucleus, to cue cessation of eating. Oral sensations such as tingling, cooling, and/or warming are provided, discouraging eating long enough for a signal of satiety to be received by the brain.

### Methods

Methods are provided for modifying eating behavior in an individual and for controlling the amount of food that the individual consumes. In the methods herein, an individual orally consumes a composition as disclosed herein after consuming a desired amount of food (e.g., the minimum amount of food that would cause satiety in the individual with the passage of time without further consumption of food). The composition may be consumed before the individual feels full and before a signal of satiety has reached the individual's brain. An individual may interrupt a habit of overeating and develop a new habit of consuming the composition before overeating has occurred. The composition may create oral sensation(s) in the individual's mouth that makes consuming further food undesirable. For example, sensations of tingling, cooling, and/or warming may be provided. In some embodiments, the composition is consumed after a meal. A “meal” may include a desired amount of food and/or number of calories. Meals are often consumed on a daily basis several times a day, such as, but not limited to, breakfast, lunch, dinner, brunch, or the like. In some embodiments, a meal includes an amount of food (e.g., a minimum amount of food) that would create satiation in an individual with the passage of time (e.g., about 10 to about 30 minutes after cessation of eating). A meal may be larger, more filling, and more varied than a snack. By consuming the composition after multiple meals (e.g., after one, two, three, or more meals over the course of a day, week, month, etc.) and on a consistent basis, the individual may acquire a habitual sense of meal completion when consuming the oral composition that lasts long enough and bridges the time until a natural biological signal of satiety reaches the brain of the individual. The composition may cue a cessation of eating and allow the individual to stop eating long enough to acquire a sense of satiety from food already consumed. In some embodiments, the sense of meal completion lasts for at least about 10 minutes, at least about 15 minutes, at least about 20 minutes, at least about 25 minutes, or at least about 30 minutes, and/or for a length of time that is sufficient for the individual to acquire a feeling of satiety without further consumption of food.

In some embodiments, the composition stimulates the third division of the trigeminal nerve. An individual may acquire a habit from consistent consumption of the composition after eating a desired amount of food such that repeated stimulation of the trigeminal nerve via the oral sensation(s) created by the composition cues the cessation of eating. Methods as described herein may be used for weight loss (reduction of body weight), treatment of obesity, or weight maintenance. The methods described herein may have the effect over time of reducing weight, decreasing body fat, increasing lean muscle mass ratio, lowering body mass, and/or reducing or eliminating symptoms and/or diseases associated with or complicated by obesity.

In methods disclosed herein, the composition for consumption after a desired amount of food may be provided in any form that is suitable for oral consumption. For example, but without limitation, the composition may be provided as a candy (e.g., a hard candy), lozenge, a toothpaste, a chewing gum, a spray, a dissolving strip, a toothpick, a dental floss, a quick shot, a tea, a dissolving drop, a beverage, a dessert, or the like.

The oral sensation(s) that cue the cessation of eating may include tingling, cooling, and/or warming sensations. In some embodiments, the composition provides a tingling sensation and either or both of cooling and warming sensations. Any compounds that are suitable for oral consumption and provide these sensations may be included in the composition. In some embodiments, a tingling sensation is provided by one or more compounds including, but not limited to, spilanthol, isobutylalkylamide, and hydroxyl-alpha sanshool, or a derivative thereof that retains the ability to provide an oral tingling sensation. In some embodiments, a cooling sensation is provided by one or more compounds, including but not limited to, menthol or a derivative thereof that retains the ability to provide an oral cooling sensation. In some embodiments, a warming sensation is provided by one or more compounds including, but not limited to, capsaicin, a capsaicinoid, shogaol, a zingerone, a gingerol, eugenol, piperine, and allyl isothiocyanate, or a derivative thereof that retains the ability to provide an oral warming sensation.

The oral sensations tingling, cooling, and/or warming (e.g., tingling and at least one (i.e., either or both) of cooling and warming, for example, tingling and cooling) may be provided in the same composition or in separate compositions that are consumed simultaneously or sequentially by the individual.

In methods described herein, the composition consumed by the individual contains amounts of the compound(s) providing tingling, cooling, and/or warming sensations sufficient to deter further eating by the individual (e.g., sufficient to deter further eating for a period of time at least until a signal of satiety has reached the brain of the individual).

In some embodiments, the composition is provided as a lozenge or candy to be consumed after a meal. The lozenge or candy may optionally include layers with one or more compounds as described herein in each layer. In one embodiment, the lozenge or candy includes an outer layer or compound coating that includes one or more sweetener. In some embodiments, the lozenge or candy contains compounds that impart tingling, cooling, and/or warming sensations (for example, tingling and cooling and/or warming, e.g., tingling and cooling mixed together in a core composition, surrounded by an outer layer or compound coating that includes one or more sweetener.

In some embodiments, the composition further includes one or more flavoring that does not provide a tingling, cooling, or warming sensation. Examples of such flavorings include, but are not limited to, chocolate, cocoa, coffee, cinnamon, and lime and/or other fruit flavors. In some embodiments, the flavoring is in an outer layer or compound coating of the composition, and may optionally formulated in conjunction with one or more sweetener(s).

In some embodiments, an outer layer or coating contains at least one sweetener compound and at least one flavoring. In one embodiment, the outer layer or coating contains sweetened chocolate.

In some embodiments, the composition further includes one or more additive that is not a flavoring and does not provide a tingling, cooling, or warming sensation. In some embodiments, the additive may provide a stimulant or beneficial effect to the individual. Examples of additives include, but are not limited to, caffeine, vitamins, calcium, and probiotics.

The methods described herein may be used to assist an individual in modifying their behavior, helping the individual to break a habit of overeating by substituting a habit of ending a meal at an appropriate time point before overeating has occurred. “Overeating” may refer to, but is not limited to, consumption of an amount of food that is greater than the amount of food that would cause satiety with the passage of time and/or consumption of an amount of food that is in excess of the amount of food required to satisfy the energy needs of an individual. The individual is conscious of the time at which the composition must be consumed, e.g., after a desired amount of food has been consumed (e.g., an amount of food in portion sizes and/or the minimum amount of food that would create satiation in the individual with the passage of time) and consciously aware of the need to end their meal at the appropriate time before overeating has occurred. The affirmative action and conscious decision to end a meal by consuming the composition helps to reduce or eliminate overeating by the individual. The individual breaks the cycle of eating by consuming the composition. This creates an interruption in eating and overrides the previous eating experience of the meal, thus altering the behavior of the individual from eating to cessation of eating.

In embodiments in which the composition is in a dissolvable form, such as a lozenge or candy, the individual may break the cycle of chewing by letting the composition dissolve in their mouth over time. The length of time that the composition takes to dissolve may be part or all of the time necessary to bridge the time between cessation of eating and the feeling of satiety. The time during which the composition dissolves in the individual's mouth may provide time for the process of satiety to progress. In embodiments in which the dissolvable composition contains layers that provide different oral sensations, combinations of oral sensations, and/or flavors, the individual's awareness of the act of “non-eating” may be enhanced when experiencing the multilayer taste and sensation activity of the composition. In embodiments in which the dissolvable composition contains an outer layer with one or more sweetener, the individual rapidly receives a sweet cue to the brain's reward center. Over time and with repeated use at the end of meals, this habituates the individual to a sweet cue signaling cessation of eating and end of a meal, for example, substituting for the experience of a higher calorie dessert.

In some embodiments of the methods described herein, the aroma of an individual's meal is overwhelmed by the sensation of the oral composition. Food aroma has been theorized to drive impulsive and continued eating behaviors. The individual's sensory receptors are stimulated differently than the stimulation received from the meal, changing the taste and feel in the mouth and decreasing the desire to continue eating the previous food(s) of the meal. In some embodiments, the individual experiences an energizing oral trigeminal tactile modification when the oral composition is consumed. The tingling, cooling, and/or warming effects provided by the composition stimulate the tongue's feel for several minutes, making the thought of eating more food less appealing, providing time for the process of satiety to progress.

In some embodiments, water may be consumed after or simultaneously with consumption of the oral composition. Although not wishing to be bound by theory, the water may accelerate digestion and the release of Peptide YY, providing a signal of satiety to the brain. The water may also enhance and activate an oral cooling sensation, in embodiments in which the composition provides a cooling sensation, further indicating to the individual that they have transitioned away from eating.

After repeated use of the methods described herein, an individual may develop a habitual cueing effect from the composition's tingling, cooling, and/or warming sensations that the meal is complete and further eating is not desirable and/or necessary to satisfy hunger.

In some embodiments, methods are provided for modifying the behavior of an individual to decrease or eliminate consumption of snacks (e.g., high calorie and/or unhealthy snacks) between meals and/or to avoid consumption of a snack. A “snack” may refer to a portion of food that is smaller than a meal, typically eaten between meals. An individual may desire to reduce or eliminate the consumption of snacks between meals, especially high calorie and/or unhealthy snacks, including, but not limited to, chips, cookies, candy, crackers, doughnuts, etc. The compositions described herein may be consumed as a deterrent to snacking. In some embodiments, habitual cueing of cessation of eating is developed, by consuming a composition that provides oral stimulation (e.g., oral trigeminal stimulation) at the end of meals, as described above. When the individual in whom such a habit has been developed has the urge to eat a snack, the composition may be consumed, creating a sensation in the individual's mouth that serves as a deterrent to consuming the snack. The composition may provide a “don't eat” cue, which has been developed by repeated consumption in the context of cessation of eating at the end of meals, and which provides a distraction and/or transference from the desire to snack and assists the individual with resisting consuming a snack. In other embodiments, the habit of cessation of eating has not been developed in the individual by consumption of the composition after meals. In such an embodiment, the individual consumes the composition that provides oral stimulation (e.g., oral trigeminal stimulation) as described herein in lieu of a snack, creating a distraction and/or transference from the desire to snack, and/or creating oral sensation(s) that render snacking undesirable.

### Compositions

Compositions are provided for use in the methods disclosed herein. The compositions may signal completion of a meal when consumed by an individual. In some embodiments, the compositions are formulated for oral stimulation of the trigeminal nerve, e.g., the third division of the trigeminal nerve.

In some embodiments, the compositions herein promote sensations of tingling, cooling, and/or warming when orally consumed by an individual. In one embodiment, the composition includes one or more compounds that promote an oral sensation of tingling and one or more compounds that promote an oral sensation of cooling. In another embodiment, the composition includes one or more compounds that promote an oral sensation of tingling and an oral sensation of warming. In another embodiment, the composition includes one or more compounds that promote an oral sensation of cooling and one or more compounds that promote an oral sensation of warming. In another embodiment, the composition includes one or more compounds that promote an oral sensation of tingling, one or more compounds that promote an oral sensation of cooling, and one or more compounds that promote an oral sensation of warming.

In some embodiments, the composition includes at least one compound that provides a tingling sensation when consumed orally by an individual, including but not limited to, spilanthol (e.g., from Jambu oleoresin), isobutylalkylamide (IBA), and hydroxyl-alpha sanshool (e.g., active oil of Szechuan peppercorn), or a derivative thereof that retains the ability to provide an oral tingling sensation (e.g., a tingling sensation on the tongue of an individual).

In some embodiments, the composition includes at least one compound that provides a cooling sensation when consumed orally by an individual, including but not limited to, menthol or a derivative thereof that retains the ability to provide an oral cooling sensation (e.g., a sensation of cold or coolness on the tongue of an individual). See, e.g., www.leffingwell.com/cooler_than_menthol.htm, which is incorporated herein by reference, for nonlimiting examples of cooling agents.

In some embodiments, the composition includes at least one compound that provides a warming sensation when consumed orally by an individual, including but not limited to, capsaicin (e.g., from chiles), a capsaicinoid (e.g., from chiles), shogaol (e.g., from ginger), a zingerone (e.g., from ginger), a gingerol (e.g., from ginger), eugenol (e.g., from clove), piperine (e.g., from black pepper), and allyl isothiocyanate (e.g., from mustard or horseradish), or a derivative thereof that retains the ability to provide an oral warming sensation (e.g., a sensation of heat or warmth on the tongue of an individual).

The compositions herein may be formulated in any form that is suitable for oral consumption by an individual. Nonlimiting examples of such formulations include lozenges, candies, toothpastes, chewing gums, sprays, dissolving strips, toothpicks, dental floss, quick shots, teas, dissolving drops, beverages, and desserts.

In one embodiment, the composition is formulated as a lozenge or candy (e.g., hard candy). In some embodiments, the lozenge contains multiple layers. The lozenge may optionally contain a layer (e.g., an outer layer) with a different flavor and/or that provides a different sensation than the tingling, cooling, and/or warming compounds described above. In one embodiment, the lozenge contains an outer layer or compound coating that provides a sweet taste sensation (e.g., dessert-like flavor). For example, one or more sweetener compounds may be included in the outer layer. The sweetener may be a sugar (e.g., a sweet flavored carbohydrate substance, for example glucose, fructose, or sucrose) or a sugar substitute that duplicates the effect of sugar in taste and may optionally be lower in caloric value than sugar. In some embodiments, different compounds or subsets of compounds that provide tingling, cooling, and/or warming sensations may be located in different layers of a lozenge. In some embodiments, different layers provide different sensations (e.g., tingling, cooling, or warming in different layers). In some embodiments, layers contain compounds that provide at least two of tingling, cooling, and warming sensations. In one embodiment, the lozenge includes at least one inner layer or a core that provides a tingling sensation and at least one of cooling and warming sensations (e.g., tingling and cooling) and at least one outer layer or compound coating that provides a sweet taste and optionally includes one or more flavoring substance(s). In some embodiments, the composition contains a mixture of compounds that impart tingling, cooling, and/or warming sensations (e.g., tingling and cooling), and optionally the mixture of compounds is in an inner core and the composition further includes an outer layer that includes one or more sweetener(s) and/or one or more flavoring(s).

In some embodiments, the composition includes at least one flavoring substance, in addition to compounds that provide the tingling, cooling, and/or warming sensations described above. For example, the flavoring substance may be a compound that provides an oral flavor sensation but does not provide a tingling, cooling, and/or warming sensation. Examples of flavorings include, but are not limited to, chocolate, cocoa, coffee, cinnamon, and lime and/or other fruit flavors. The flavoring substance(s) may be in the same or different layer or portion of the composition than the compounds that provide tingling, cooling, and/or warming sensations. Alternatively, the flavoring may be in a separate composition and may be consumed simultaneously or sequentially (before or after) with respect to the composition that provides tingling, cooling, and/or warming sensations.

In some embodiments, an outer layer or coating contains at least one sweetener compound and at least one flavoring. In one embodiment, the outer layer or coating contains sweetened chocolate.

In some embodiments, the composition includes at least one additive substance, in addition to compounds that provide the tingling, cooling, and/or warming sensations described above. For example, the additive substance may be a compound that provides a beneficial and/or stimulatory effect to an individual but does not provide a tingling, cooling, and/or warming sensation. Examples of additives include, but are not limited to, caffeine, vitamins, calcium, and probiotics. The additive substance(s) may be in the same or different layer or portion of the composition than the compounds that provide tingling, cooling, and/or warming sensations. Alternatively, the additive substance(s) may be in a separate composition and may be consumed simultaneously or sequentially (before or after) with respect to the composition that provides tingling, cooling, and/or warming sensations.

The compositions disclosed herein may include other components and ingredients that provide stability and/or make the composition suitable for oral consumption by an individual and/or for aesthetic purposes, including but not limited to, fillers, binders, lubricants, stabilizers, buffers, bulking agents, viscosity enhancers or reducers, surfactants, and/or pigments.

Use of any of the compositions disclosed herein in a method for modifying eating behavior, a method for weight loss, a method for treating obesity, or a method for maintaining a desired weight are provided.

### Systems and Kits

Systems for use in any of the methods described herein, such as methods for modifying eating behavior, methods for weight loss, methods for treatment of obesity, and methods for maintaining a desired body weight, are provided. Systems herein include one or more portions or “dosage forms” of the compositions described herein for consumption by an individual after a meal or after a desired amount of food has been consumed. A dosage form of a composition includes an amount of the composition suitable for consumption after a meal as described herein, for example, containing substances that provide oral stimulatory effects such as tingling, cooling, and/or warming, in amounts that are sufficient to deter further eating by an individual after consumption of the composition, e.g., sufficient to deter further eating long enough for a signal of satiety to reach the individual's brain. The system may optionally include instructions for use in a method as described herein, either in printed form, in electronic form such as on a compact disc or other magnetic media, or provided as information as to how to obtain the instructions from another source such as over the Internet, such as a website address, and/or in a mobile application.

Kits are also provided that include one or more dosage forms of the compositions as described herein in packaging, and optionally further including instructions for use as described above. The dosage forms may be individually packaged, or multiple dosage forms may be contained within a single package, such as a box, pouch, or bag. In some embodiments, the dosage forms may be individually wrapped for ease of use by an individual.

The following examples are intended to illustrate, but not limit, the invention.

### EXAMPLES

### Example 1

An In-Home Use Product Test (IHUT) was conducted to test the effectiveness of the compositions and methods described herein in promoting weight management, weight loss, and other parameters.

Study participants self-administered lozenges that imparted tingling and cooling signaling properties when dissolved orally, as described herein, over a four-week period. IHUT participants were individuals who were interested in weight management and control. 82 participants were recruited, of which 77 stayed on the study from weeks 1 to 4. Participants were 82.9% female, with the median age for participants ranging from 41 to 50 years, and almost all were currently trying to manage their weight at the initiation of the study. Two-thirds of the participants were dieting. Median targeted weight reduction was from 30 to 39 pounds.

Participants were told to allow a lozenge to dissolve in the mouth on a regular basis at the end of meals when they knew they had had enough to eat, but wanted to continue eating. They were also told to use the lozenges in place of a snack to bridge the gap between meals when they had they urge to eat something and the normal inclination would be to instead consume a high calorie treat.

“Full Treats” and “Boosters” were provided in a variety of flavors. “Full Treats” were lozenges that contained an outer “reward” layer, which provided a sweet, dessert-like taste, which individuals typically associate with the end of a meal, and an inner active core which provided tingling and cooling properties after the reward layer dissolved in the mouth. “Boosters” contained only the core, with no outer reward layer. Participants were instructed to use Full Treats and Boosters as desired, to determine which worked best for them after meals and for snack avoidance.

IHUT participants consumed the lozenges in a variety of contexts, including after meals, instead of snacks, and/or after dessert. After dinner and after lunch were the two most frequent occasions when the product was used, followed by mid-morning in place of a snack. Seven out of 10 participants used the product to both avoid snacking and stop further eating after a meal; only a small proportion used it for either purpose alone. The number of lozenges consumed ranged from 1 to 5 per day. Two to three per day were typically consumed.

Roughly 3 out of 4 participants believed that the product helped them consume fewer calories, earning positive ratings on efficacy. Perception of effectiveness of the taste sensations to signal cessation of eating is shown in Table 1. Perception of reduction in consumed calories is shown in Table 2.

TABLE 1
Perception of effectiveness in promoting cessation of eating

| Week no. | Mean score on 1 to 10 scale |
|---|---|
| 1 | 6.92 |
| 2 | 6.94 |
| 3 | 6.78 |
| 4 | 6.62 |

TABLE 2
Perception of effectiveness in reduction of calories consumed

| Week no. | Yes (%) | No (%) |
|---|---|---|
| 1 | 76 | 24 |
| 2 | 74 | 26 |
| 3 | 74 | 26 |
| 4 | 77 | 23 |

The majority of participants stopped further eating after consuming the study lozenges, avoided snacking, and lost weight. 79% of study participants used the study lozenges to stop eating and 88% of participants used the lozenges to avoid snacking For example, during week three, 79% stopped further eating, 88% avoided snacking, and 82% lost weight.

In week 4, participants were asked to rate the lozenges as an invaluable tool to make existing diets more effective, on a scale of 1 to 10, with 10 being the most invaluable. The average rating was 6.4 and the median was 7.0. Participants were also asked to rate how effective the lozenges were in helping them feel in control of how much they ate, on a scale of 1 to 10, with 10 being the most invaluable. The average rating was 6.7 and the median was 8.0.

During the four-week product test period, the median number of pounds lost was 1 to 5 pounds. Weight loss over the course of the study is shown in Table 3.

TABLE 3
Weight loss

| Weight lost (lbs.) | Male | Female | Total |
|---|---|---|---|
| 10-19 | 3 | 9 | 12 |
| 5-9 | 2 | 16 | 18 |
| 1-5 | 6 | 26 | 32 |
| 0 | 2 | 12 | 42 |
| Total | 13 | 63 | 76 |

Although the foregoing invention has been described in some detail by way of illustration and examples for purposes of clarity of understanding, it will be apparent to those skilled in the art that certain changes and modifications may be practiced without departing from the spirit and scope of the invention, which is delineated in the appended claims. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

1. A method for modifying eating behavior in an individual, said method comprising:
orally consuming a composition after consuming an amount of food, wherein the composition causes at least one oral sensation in the individual that makes consuming a further amount of food undesirable, wherein the individual acquires a habitual sense of meal completion after consuming said composition after each meal for a period of time.

2.-4. (canceled)

5. A method according to claim 1, wherein the sense of meal completion lasts at least until the individual acquires a feeling of satiety without further consumption of food.

6. A method according to claim 1, wherein the composition stimulates the third division of the trigeminal nerve, and wherein said stimulation of the trigeminal nerve cues the cessation of eating.

7.-8. (canceled)

9. A method according to claim 1, wherein the oral sensation comprises at least two sensations selected from tingling, cooling, and warming.

10.-11. (canceled)

12. A method according to claim 9, wherein the oral sensation comprises a tingling sensation that is provided by at least one compound selected from spilanthol, isobutylalkylamide, and hydroxyl-alpha sanshool.

13. A method according to claim 9, wherein the oral sensation comprises a cooling sensation that is provided by menthol or a derivative thereof.

14. A method according to claim 9, wherein the oral sensation comprises a warming sensation that is provided by at least one compound selected from capsaicin, a capsaicinoid, shogaol, a zingerone, a gingergol, eugenol, piperine, and allyl isothiocyanate.

15.-20. (canceled)

21. A method according to claim 1, wherein the method comprises a method for weight loss in an individual in need thereof, comprising consuming the composition after the individual has consumed a desired amount of food, wherein the individual ceases eating earlier than they would in the absence of consumption of the composition, and wherein consuming the composition after eating causes the individual to consume less food and lose weight.

22.-25. (canceled)

26. A method according to claim 1, wherein the method comprises a method for maintaining a desired weight in an individual in need thereof, comprising consuming the composition after the individual has consumed a desired amount of food, wherein the consumption of the composition is continued after meals for a period of time until a habit is established in the individual that a meal is completed after the composition has been consumed, and wherein establishment of the habit causes the individual to maintain their weight in a desired weight range.

27. A method according to claim 1, wherein the method comprises a method for reducing or eliminating a desire to consume a snack between meals, wherein the individual consumes the composition when the desire for a snack occurs, and wherein the oral sensation caused by consumption of the composition reduces or eliminates the desire to consume the snack.

28. A composition for signaling completion of a meal, said composition comprising compounds that provide at least two oral sensations selected from tingling, cooling, and warming when consumed orally by an individual.

29.-31. (canceled)

32. A composition according to claim 28, wherein the oral sensation comprises a tingling sensation that is provided by at least one compound selected from spilanthol, isobutylalkylamide, and hydroxyl-alpha sanshool.

33. A composition according to claim 28, wherein the oral sensation comprises a cooling sensation that is provided by menthol or a derivative thereof.

34. A composition according to claim 28, wherein the oral sensation comprises a warming sensation that is provided by at least one compound selected from capsaicin, a capsaicinoid, shogaol, a zingerone, a gingergol, eugenol, piperine, and allyl isothiocyanate.

35. A composition according to claim 28, wherein the composition is formulated in a lozenge, a candy, a toothpaste, a chewing gum, a spray, a dissolving strip, a toothpick, a dental floss, a quick shot, a tea, a dissolving drop, a beverage, or a dessert.

36. A composition according to claim 28, wherein the composition is formulated in a lozenge with an outer layer or coating that comprises at least one sweetener.

37. A composition according to claim 28, wherein the composition further comprises at least one flavoring that does not provide a tingling, cooling, or warming sensation.

38. A composition according to claim 37, wherein at least one flavoring is selected from chocolate, cocoa, coffee, cinnamon, and lime and/or other fruit flavors.

39. A composition according to claim 28, wherein the composition further comprises at least one additive that does not provide a tingling, cooling, or warming sensation.

40. Original) A composition according to claim 39, wherein at least one additive is selected from caffeine, a vitamin, calcium, and a probiotic.

41. (canceled)
